(19) United States
(12) Patent Application Publication
(10) Pub. No.: US 2025/0353896 A1
(43) Pub. Date: Nov. 20, 2025

(54) PROCESS FOR THE PRODUCTION OF IRISIN, ITS FORMULATIONS AND ITS ADMINISTRATION ROUTES

(71) Applicant: Universita' degli Studi di Bari Aldo Moro, Bari (IT)

(72) Inventors: Maria Grano, Bari (BA) (IT); Silvia Concetta Colucci, Bari (BA) (IT); Graziana Colaianni, Bari (BA) (IT); Roberta Zerlotin, Bari (BA) (IT); Patrizia Pignataro, Bari (BA) (IT)

(73) Assignee: Universita' degli Studi di Bari Aldo Moro, Bari (IT)

(21) Appl. No.: 18/861,953

(22) PCT Filed: May 11, 2023
(86) PCT No.: PCT/IB2023/054861

§ 371 Date: Oct. 31, 2024

(30) Foreign Application Priority Data
May 11, 2022 (IT) 102022000009782

(51) Int. Cl. C07K 14/78; A61K 9/1272; A61K 38/39; C12N 15/74; C12N 15/82

(52) U.S. Cl. CPC C07K 14/78; A61K 9/1272; A61K 38/39; C12N 15/743; C12N 15/8205; C12N 15/8257

(57) ABSTRACT

The present invention relates to a process for the production of irisin, comprising the following steps: providing an expression vector comprising a nucleotide sequence coding for said irisin; inserting said expression vector into at least one first bacterium of the Agrobacterium genus, thus obtaining a first bacterium of the Agrobacterium genus comprising said expression vector; treating at least one plant material with said first bacterium comprising said expression vector, thus obtaining a treated plant material; cultivating said treated plant material, so that said treated plant material expresses said irisin; and extracting said irisin. The present invention further relates to irisin included in liposomes, its use as a medicament and its administration routes, a synthetic gene comprising a sequence coding for irisin and additional elements.

### TECHNICAL FIELD

The present invention relates to a process for the production of irisin, in particular recombinant irisin, in organisms of plant origin. The present invention further relates to formulations of irisin and its routes of administration.

### STATE OF THE ART

Irisin, a molecule produced by the muscles during physical exercise, is a myokine released upon cleavage of the membrane protein containing the type III domain of fibronectin (FNDC5). It was initially described for its ability to induce trans-differentiation of white adipocytes into brown ones but subsequent studies have highlighted more far-reaching effects of irisin on other tissues and organs. Among these effects are of great clinical relevance: the ability, exerted by low doses of irisin on the musculoskeletal system, to prevent and cure osteoporosis and muscle atrophy; its role in regulating energy metabolism by attenuating the insulin resistance; and the ability to protect memory and the cognitive decline in neurodegenerative diseases such as Alzheimer's disease.

Thus, irisin is emerging as a molecular key to metabolic diseases and other disorders known to manifest improvements following physical exercise.

Translating the above mentioned results, obtained mainly in mouse models, to humans could lead to the development of a drug that would represent a highly innovative exercise-mimetic therapeutic strategy and would have several applications in the medical field. However, further steps of preclinical studies, with particular reference to the regulatory pre-clinic prodromal to human clinical trials, involve experimental studies in large animal models and therefore require huge amounts of protein, which must be produced according to Good Manufacturing Practice (“GMP”).

Such a development first requires large-scale, cost-effective production of the molecule that is as safe as possible for patients.

Recombinant DNA technology has made possible the production of irisin in Escherichia coli and in Chinese Hamster Ovary cells (“CHO” cells). However, the expression of recombinant proteins in such systems suffers from a number of drawbacks, including low yield, the complexity of the protocols to be applied on a large scale, the possible presence of immunogenic endotoxins and very high costs. Such drawbacks, together with high costs, make the development of a possible irisin-based drug, the use of which could affect high numbers of patients with various diseases, particularly complex.

Therefore, it is necessary to provide a process for the production of irisin, which allows irisin, in high amount, to be obtained easily, quickly and inexpensively.

In addition to the technical problems associated with its production, there are a number of issues related to the administration of irisin. For example, in its native form, irisin may not be administered orally because it would be digested at the gastric and/or intestinal level by the proteases present in the various tracts of the gastrointestinal tract, and this is a limitation for the compliance of possible patients approaching a chronic irisin-based therapy.

### OBJECTS OF THE INVENTION

Object of the present invention is to provide a process for the production of irisin, which allows irisin, in high amount, to be obtained easily and quickly.

Further object of the present invention is to provide a process for the production of irisin, having high yield and low cost.

Still object of the present invention is to provide a formulation of irisin which allows improving the pharmacodynamic and pharmacokinetic properties of such molecule and patient compliance when it comes to human trials.

Yet again object of the present invention is to provide a formulation of irisin which may be administered safely, easily and quickly.

Still object of the present invention is to provide a synthetic gene which allows high expression levels of irisin to be achieved.

### DESCRIPTION OF THE INVENTION

The purposes above, as well as other purposes, are achieved by the object of the present invention, namely by a process for the production of irisin, in particular recombinant irisin, comprising the following steps:
a) providing at least one expression vector, preferably at least one plasmid, comprising a nucleotide sequence coding for irisin;
b) inserting said expression vector into at least one bacterium of the Agrobacterium genus, thus obtaining a bacterium of the Agrobacterium genus comprising said expression vector;
c) treating at least one plant material with said bacterium comprising said expression vector, thus obtaining a treated plant material;
d) cultivating said treated plant material, so that said treated plant material expresses said irisin;
e) extracting said irisin from said plant material.

In fact, it has been observed that plant-derived organisms are capable of expressing irisin, in particular recombinant irisin, for example in non-glycosylated or mildly glycosylated form, but with yields significantly higher than those achievable by known techniques.

Furthermore, plants have a number of advantages that make them attractive for the production of recombinant proteins, in particular for pharmacological purposes. For example, they may be easily cultivated on a large scale, in small greenhouses, and be free of immunogenic bacterial endotoxins which are a major difficulty in purification processes of the proteins produced by E. coli.

Furthermore, when compared with conventional expression systems, such as the microbial fermentation and mammalian cell cultures, the plant production systems are inexpensive, may be easily implemented on an industrial scale and are found to be free of pathogens hazardous to human health.

By the term “recombinant irisin” is meant here to refer to irisin obtained by transcription and translation of a recombinant DNA fragment inserted within a host organism.

By the term “recombinant DNA” is meant here to refer to a DNA sequence obtained artificially by combining genetic material of different origins, as may occur, for example, in the case of a plasmid containing a gene of interest.

In embodiments of the present invention, irisin is in its non-glycosylated form or in its mildly glycosylated form.

The production of non-glycosylated or mildly glycosylated irisin has a number of advantages over the production of glycosylated irisin (i.e., compared with irisin production in which most of the molecules are in a glycosylated form). In fact, it has been surprisingly observed that the yield of the production of non-glycosylated or mildly glycosylated irisin is particularly high compared with the yield of the production of glycosylated irisin (i.e., compared with irisin production in which most of the molecules are in a glycosylated form). In particular, western blot and ELISA assays have made it possible to observe how the methods adapted to produce glycosylated irisin (i.e., compared with irisin production in which most of the molecules are in a glycosylated form) do not allow production of irisin in significant amounts.

For example, the production of non-glycosylated irisin may be achieved by using a gene sequence that does not include the coding sequence for the signal peptide capable of directing irisin to the endoplasmic reticulum.

For example, the production of mildly glycosylated irisin may be achieved by using a gene sequence that includes a sequence coding for a KDEL tag, which allows the retention of irisin in the endoplasmic reticulum.

The nucleotide sequence coding for irisin is currently commercially available and may be obtained in the form of a plasmid, preferably a pUC57 plasmid, comprising such nucleotide sequence (e.g., from GenScript, Piscataway, NJ).

The sequence coding for irisin may be isolated from the original plasmid by the use of appropriate restriction enzymes, for example, BamHI and XmaI restriction enzymes.

Before starting to insert the nucleotide sequence coding for irisin into an expression vector adapted to be inserted into plant material, such sequence may be inserted into an intermediate vector (or amplification vector) in order to be amplified, that is, in order to produce a large number of copies of such sequence. The intermediate vector containing the sequence coding for irisin is used to transform Escherichia coli cells. The Escherichia coli cells are then cultured, and at the end of the cell growth, the intermediate vector containing the sequence coding for irisin is purified.

Therefore, in embodiments, the process according to the invention comprises a preliminary step, i.e., before step a), in which an intermediate vector (or amplification vector) comprising the nucleotide sequence coding for irisin is inserted into Escherichia coli and amplified by culture of said Escherichia coli.

Preferably, the intermediate vector is a plasmid, more preferably the pGEM-NOS plasmid.

Following the preliminary amplification step described above, the sequence coding for irisin is inserted into the expression vector, preferably together with a sequence with a plant terminator function, preferably the Nos-ter terminator. The terminator is a sequence capable of stalling the transcription of a gene and allows the integration of the expression vector into the plant genome. The Nos-ter terminator is a known terminator, per se, in art. In embodiments, the sequence coding for irisin is inserted into the expression vector, preferably together with a sequence with a plant terminator function, optionally after being amplified in E. coli.

After being amplified in E. coli, the intermediate vector containing the sequence coding for irisin is purified and the sequence coding for irisin, preferably together with a sequence with a plant terminator function (e.g., Nos-ter) is extracted from the intermediate vector and inserted into the expression vector.

In embodiments, the expression vector is a plasmid, preferably, the pBIΩ plasmid. Once the nucleotide sequence coding for irisin is inserted, that plasmid is referred to as pBIΩ-IRS.

In embodiments, the expression vector is a plasmid, preferably the modified pJL-TRBO plasmid.

By the expression “modified pJL-TRBO plasmid” is meant here to refer to a pJL-TRBO plasmid in which the restriction sites pBluescriptKS, Pvu I, GFP-R, BstZ17 I and GFP-F have been removed, and into which the restriction sites Sal I, Mlu I, Nhe I, Bmt I, Eco53k I, Sac I, Nco I, Nru I, Asc I-BssH II, ApaL I, AsiS I, Swa I, Kas I, Nar I, Sfo I, PluT I, Avr II have been inserted.

The sites pBluescriptKS, Pvu I, GFP-R, BstZ17 I and GFP-F, as well as Sal I, Mlu I, Nhe I, Bmt I, Eco53k I, Sac I, Nco I, Nru I, Asc I-BssH II, ApaL I, AsiS I, Swa I, Kas I, Nar I, Sfo I, PluT I, Avr II are restriction sites known, per se, in the art.

By the expression “pJL-TRBO” or the expression “unmodified pJL-TRBO” is meant here to refer to the pJL-TRBO plasmid in its original version, currently commercially available and sold by Addgene (Watertown, MA, USA).

The modified pJL-TRBO plasmid comprises 47 restriction sites, whereas the unmodified pJL-TRBO comprises 35 restriction sites.

The modified pJL-TRBO plasmid may be obtained from the unmodified pJL-TRBO plasmid by techniques known, per se, in the art.

The modified pJL-TRBO vector may be used to express irisin in plant material.

Advantageously, the use of the modified pJL-TRBO plasmid allows to achieve much higher agro-infection efficiency than the unmodified pJL-TRBO plasmid. Furthermore, the use of the modified pJL-TRBO plasmid allows to achieve higher levels of the expression of irisin than the unmodified pJL-TRBO plasmid. In addition, the use of the modified pJL-TRBO plasmid allows the formation of viral particles during the infection-replication cycle to be avoided.

In embodiments, the expression vector is selected from the pBIΩ plasmid and the modified pJL-TRBO plasmid.

Furthermore, the modified pJL-TRBO plasmid includes a 35S promoter from CAMV (cauliflower mosaic virus) that allows the expression of high levels of proteins in the plant, a replication origin site, a gene for kanamycin antibiotic resistance, a gene of the replication initiation protein (trfA), the 5′-leader sequence (named Omega) of the tobacco mosaic virus (TMV), a “Left border repeat of T-DNA” region, a “Right border repeat of T-DNA” region and a KS primer sequence used for the amplification and sequencing of the right end of the gene.

Once the region between the border repeats of T-DNA (i.e., between the “Left border repeat of T-DNA” and “Right border repeat of T-DNA” regions) has been recognized by the plasmid in Agrobacterium, it is transferred to plant cells.

The insertion of the expression vector into at least one bacterium of the Agrobacterium genus may be done by using techniques known, per se, in the art, such as, for example, electroporation.

The plant material may be selected from a plant, or at least one part of a plant or at least one cell of said plant.

For example, the plant material may be a plant selected from tobacco, (preferably Nicotiana benthamiana), Cannabis sativa, Arthrospira platensis, Chlorella, Arabidopsis, corn, rice, soy, canola, alfalfa, sunflower, sorghum, wheat, cotton, peanut, tomato, potato, lettuce and chili pepper, at least one part of such plant or at least one cell thereof.

Preferably, the plant material is selected from a tobacco plant (preferably Nicotiana benthamiana), at least a part of such plant and at least one cell thereof.

In embodiments, the expression of recombinant proteins, for example, in Nicotiana benthamiana (tobacco) may be carried out by using plant leaves as plant material according to the invention. Advantageously, the use of the leaves as plant material allows eliminating the need for flowering, significantly reducing the potential for gene dispersal into the environment by the spread of pollen or seeds. Furthermore, tobacco is a non-food crop; this eliminates the risk of plant-produced recombinant proteins entering the food chain.

The treatment of the plant material according to step c) of the process may be carried out by immersing, preferably completely, the plant material in a solution containing one or more bacteria of the Agrobacterium genus (containing, in turn, the expression vectors) and applying vacuum. In embodiments, the immersion of the plant material in the solution containing Agrobacterium, preferably inside a dryer, takes only a few minutes. For example, the immersion of the plant material in the solution containing Agrobacterium may take from 5 to 15 minutes. Preferably, vacuum is applied, and once it reaches about 10 mm Hg, it is quickly released.

Step c) of the process according to the invention may be defined as an agro-infiltration step.

In embodiments, step c) of the process of the invention further comprises the step of treating the plant material with at least one second bacterium of the Agrobacterium genus, in which such second bacterium comprises an expression vector comprising a gene adapted to prevent the silencing of the expression of irisin in the treated plant material.

In embodiments, in step c) of the process of the invention, the plant material is treated with a mixture comprising a first bacterium comprising an expression vector comprising a nucleotide sequence coding for irisin, and a second bacterium comprising a gene adapted to prevent the silencing of the expression of irisin in the treated plant material.

Advantageously, the use of a gene adapted to prevent the silencing of the expression of irisin in the treated plant material allows to inhibit the fragmentation and silencing of the foreign gene (in this case, the irisin gene) by plant cells which, in the presence of an overproduced foreign transcript, i.e., produced in significantly greater amounts than the transcripts derived from endogenous genes of the host plant, in this case the irisin transcript, could activate such defense mechanisms.

Preferably, the gene adapted to prevent the silencing of the expression of said irisin is the P19 gene.

The P19 gene codes for a protein referred to as “p19 RNA silencing suppressor”. The p19 protein is able to specifically bind 19- to 21-nucleotide double-stranded RNAs that function as small interfering RNAs (siRNAs) in the RNA silencing system in plant cells. By sequestering such siRNAs, p19 suppresses the exogenous RNA silencing.

Preferably, the bacterium of the Agrobacterium genus is Agrobacterium tumefaciens.

Preferably, the nucleotide sequence coding for irisin is

(SEQ. ID. NO. 2)
GGATCCGATTCTCCTTCAGCTCCAGTTAATGTTACAGTTAGACATCTTAA
GGCTAATTCTGCTGTTGTTTCATGGGATGTTTTGGAAGATGAGGTTGTTA
TTGGTTTTGCTATCTCTCAACAGAAGAAAGATGTTAGAATGCTTAGGTTC
ATCCAAGAAGTTAACACTACAACTAGGTCTTGTGCTCTTTGGGATTTGGA
AGAGGATACAGAGTACATCGTTCATGTTCAGGCTATCTCAATCCAAGGAC
AGTCTCCTGCTTCAGAACCAGTTTTGTTTAAAACTCCTAGGGAGGCTGAG
AAAATGGCAAGTAAAAACAAGGATGAGGTGACAATGAAAGAGTGATAGC
CCGGG.

The nucleotide sequence SEQ. ID. NO. 2 includes a restriction site for the BamHI enzyme (the first underlined 6 nucleotides) and a restriction site for the SmaI enzyme (the last underlined 6 nucleotides). Highlighted in bold are 2 stop codons, i.e., the 6 nucleotides immediately preceding the restriction site for the SmaI enzyme.

In embodiments, the nucleotide sequence coding for irisin is included in a gene (in particular a synthetic gene) comprising, in addition to the sequence coding for irisin, a polynucleotide sequence coding for an N-terminal signal peptide for the secretion from the disulfide isomerase of Medicago sativa alfalfa; a sequence coding for the thrombin cleavage site, followed by a sequence coding for a tail of 8 histidine residues (8-His tag) and a sequence coding for a KDEL-terminal tail that allows the retention of the protein in the endoplasmic reticulum. The nucleotide sequence coding for irisin is located between the polynucleotide sequence coding for an N-terminal signal peptide for the secretion from the disulfide isomerase of Medicago sativa alfalfa and the sequence coding for the thrombin cleavage site.

Therefore, in embodiments, the expression vector comprising a nucleotide sequence coding for irisin may comprise a gene (in particular a synthetic gene) comprising: a polynucleotide sequence coding for an N-terminal signal peptide for the secretion from the disulfide isomerase of Medicago sativa alfalfa; a sequence coding for irisin, a sequence coding for the thrombin cleavage site, a sequence coding for a tail of 8 histidine residues (tag 8-His) and a sequence coding for a KDEL-terminal tail.

Therefore, another object of the present invention is a gene (i.e. a polynucleotide sequence, in particular a synthetic gene) comprising a polynucleotide sequence coding for an N-terminal signal peptide for the secretion from the disulfide isomerase of Medicago sativa alfalfa; a sequence coding for irisin, a sequence coding for the thrombin cleavage site, a sequence coding for a tail of 8 histidine residues (tag 8-His) and a sequence coding for a KDEL-terminal tail, fused to each other.

The use of such a synthetic gene allows to obtain a polypeptide (in particular, a synthetic peptide) constituted by: an N-terminal signal peptide for the secretion from the disulfide isomerase of Medicago sativa alfalfa, the amino acid sequence of the irisin protein, the thrombin cleavage site followed by an 8-His tag (i.e., a tail of 8 histidine residues) and a KDEL-terminal tag (i.e., a tail comprised of the residues lysine, aspartic acid, glutamic acid and leucine).

Advantageously, the use of such synthetic gene allows to achieve particularly high levels of irisin expression.

The N-terminal signal peptide for the secretion from the disulfide isomerase of Medicago sativa alfalfa allows the synthesized polypeptide to be directed to the secretory pathway. The N-terminal signaling peptide of the disulfide isomerase is introduced at the 5′ end of the coding sequence for irisin and may be removed enzymatically, for example, within the same plant cells in which the polypeptide is expressed.

The thrombin cleavage site is the site in which shearing by thrombin occurs, resulting in the removal of the 8-His tail and the KDEL tail.

The 8-His tag is used for the extraction and purification of irisin.

The KDEL-terminal tag (i.e., a tail comprised of the residues lysine, aspartic acid, glutamic acid and leucine) allows the retention of the protein in the endoplasmic reticulum. Advantageously, the use of the KDEL tail contributes to a 2- to 10-fold increase in the expression of recombinant proteins. The KDEL tag may be removed enzymatically, for example, after the extraction of irisin from the plant material by thrombin, along with the histidine tail.

For example, the nucleotide sequence coding for irisin according to the present invention is included in a gene (in particular a synthetic gene) having the following sequence:

(SEQ. ID NO. 8)
atggctaagaatgttgctatttttggacttcttttttctcttcttgttctt
gttccatctcaaatttttgctgattctccttcagctccagttaatgttaca
gttagacatcttaaggctaattctgctgttgtttcatgggatgttttggaa
gatgaggttgttattggttttgctatctctcaacagaagaaagatgttaga
atgcttaggttcatccaagaagttaacactacaactaggtcttgtgctctt
tgggatttggaagaggatacagagtacatcgttcatgttcaggctatctca
atccaaggacagtctcctgcttcagaaccagttttgtttaaaactcctagg
gaggctgagaaaatggcaagtaaaaacaaggatgaggtgacaatgaaagag
cttgttccaagaggatctcatcatcatcatcatcatcatcat
aaggatgaactt.

The polynucleotide sequence SEQ. ID. NO. 8 includes the polynucleotide sequence of irisin. Highlighted in bold is the sequence coding for the N-terminal signal peptide for the secretion from the disulfide isomerase of Medicago sativa alfalfa; highlighted with single underline is the sequence coding for the thrombin cleavage site; highlighted with double underline is the sequence coding for the 8-His tag; and highlighted with the combination of bold and single underline is the sequence coding for the KDEL tag.

Preferably, the polypeptide that is obtained by using said synthetic gene has the following sequence:

(SEQ. ID NO. 9)
MAKNVAIFGLLFSLLVLVPSQIFADSPSAPVNVTVRHLKANSAVVSWDVL
EDEVVIGFAISQQKKDVRMLRFIQEVNTTTRSCALWDLEEDTEYIVHVQA
ISIQGQSPASEPVLFKTPREAEKMASKNKDEVTMKELVPRGSHHHHHHHH
KDEL

The amino acid sequence SEQ. ID. NO. 9 includes the amino acid sequence of irisin. Highlighted in bold is the N-terminal signal peptide for the secretion from the disulfide isomerase of Medicago sativa alfalfa; highlighted with single underline is the thrombin cleavage site; highlighted with double underline is the 8-His tag; and highlighted with the combination of bold and single underline is the KDEL tag.

In embodiments, when irisin is produced by using the synthetic gene of the invention, irisin may exhibit mild glycosylation, that is, be mildly glycosylated.

By the expression “mild glycosylation”, it is meant to refer to a partial glycosylation, in which about 10% of irisin molecules are glycosylated. For example, by the term “mild glycosylation” we may refer to a partial glycosylation in which the glycosylated irisin molecules are in an amount lower than or equal to 10% to the number of total irisin molecules.

In embodiments, mildly glycosylated irisin is treated, for example enzymatically, to remove the glucose groups, yielding non-glycosylated irisin.

In embodiments, irisin extracted from the plant material may be partially glycosylated (in particular mildly glycosylated, in which about 10% or less of the irisin molecules are glycosylated) and the process further includes a step of removing the glycosylation (i.e., the glucose groups), for example enzymatically. The removal of the glycosylation may be performed by techniques known, per se, in the art.

The cultivation of the processed plant material, according to step d) of the process of the invention, may be carried out for a variable time, depending on the plant used, preferably in a greenhouse. For example, if the plant is a tobacco plant, the processed plant material is cultivated for 2 to 7 days, preferably 6 to 7 days.

The extraction of irisin synthesized from the plant material, according to step e) of the process of the invention, may be carried out by techniques known, per se, in the art. For example, the extraction may be carried out by using the PBS buffer supplemented with protease inhibitors.

According to embodiments of the present invention, irisin does not require a histidine tail (“His-tag”) for its purification. Advantageously, the absence of the His-tag tail allows greatly reducing costs. In fact, the His-tag histidine tail must be removed, for example, by enzymatic treatment with Enterokinase (EK) before irisin may be administered in vivo, implying a significant increase in cost compared with processes that do not involve the use of His-tags in production and purification of irisin. Furthermore, it was observed that recombinant irisin with His-Tag at the C-terminal position completely loses important biological functions (for example, its ability to stimulate osteoblast differentiation), confirming the need to remove the histidine tail to allow irisin to perform important biological functions.

In embodiments, for example when irisin is produced by using the synthetic gene according to the present invention, the his-tag tail may be removed by enzymatic treatment with thrombin. Advantageously, the presence of a histidine tail allows very rapid, efficient and accurate purification of irisin, for example by immobilized metal ion affinity chromatography (IMAC).

In embodiments, the expression vector is the pJL-TRBO plasmid in which the pBluescriptKS, Pvu I, GFP-R, BstZ17 I and GFP-F restriction sites have been removed and into which Sal I, Mlu I, Nhe I, Bmt I, Eco53k I, Sac I, Nco I, Nru I, Asc I-BssH II, ApaL I, AsiS I, Swa I, Kas I, Nar I, Sfo I, PluT I, Avr II the restriction sites have been inserted and in which said expression vector comprises a gene comprising: a polynucleotide sequence coding for an N-terminal signal peptide for the secretion from the disulfide isomerase of Medicago sativa alfalfa; a sequence coding for irisin; a sequence coding for the thrombin cleavage site; a sequence coding for an 8-residue histidine tail (8-His tag) and a sequence coding for a KDEL-terminal tail.

Advantageously, the use of the modified pJL-TRBO expression vector and the inclusion of the sequence coding for irisin in a gene further comprising a polynucleotide sequence coding for an N-terminal signal peptide for the secretion from the disulfide isomerase of Medicago sativa alfalfa; a sequence coding for the thrombin cleavage site, a sequence coding for an 8-residue histidine tail (8-His tag) and a sequence coding for a KDEL-terminal tail, allows a yield of irisin production greater than 5 mg of protein per gram of fresh plant material to be obtained.

In embodiments, the expression vector is the modified pJL-TRBO plasmid and the nucleotide sequence coding for irisin is included in a gene (in particular a synthetic gene) having the sequence:

(SEQ. ID NO. 8)
atggctaagaatgttgctatttttggacttcttttttctcttcttgttctt
gttccatctcaaatttttgctgattctccttcagctccagttaatgttaca
gttagacatcttaaggctaattctgctgttgtttcatgggatgttttggaa
gatgaggttgttattggttttgctatctctcaacagaagaaagatgttaga
atgcttaggttcatccaagaagttaacactacaactaggtcttgtgctctt
tgggatttggaagaggatacagagtacatcgttcatgttcaggctatctca
atccaaggacagtctcctgcttcagaaccagttttgtttaaaactcctagg
gaggctgagaaaatggcaagtaaaaacaaggatgaggtgacaatgaaagag
cttgttccaagaggatctcatcatcatcatcatcatcatcataaggatgaa
ctt.

Another object of the present invention is irisin included in liposomes. In other words, the present invention relates to liposomes (at least one liposome) that include irisin, preferably that encapsulate irisin.

In the present description, the term “liposome” is intended to denote a vesicle constituted by at least one lipid bilayer and an aqueous solution core encapsulated within the lipid bilayer. The lipids constituting the liposome bilayer (or liposome-forming lipids) may comprise mixtures comprised primarily of phospholipids, such as phosphatidylcholine and cholesterol. Preferably, irisin is encapsulated within said liposomes.

Irisin included in liposomes may be non-glycosylated irisin.

In embodiments, irisin included in the liposomes is produced in a plant material, that is, as obtained according to the process of the invention.

For example, the liposomes containing irisin may be prepared from a solution of soy lecithin in methanol. Irisin is added to such solution, and following this addition, the solution is sonicated. A chitosan solution is then added to the solution.

In embodiments, the liposomes comprise lecithin and chitosan and, preferably, are nanoparticles comprising lecithin and chitosan.

Advantageously, the liposomes provide a useful means to facilitate the passage of the active compounds they carry across physiological barriers, increasing the bioavailability of such active compounds. The liposomes are capable of creating packets of encapsulated active compound (in this case irisin) that is physically isolated from the surrounding environment and protected from any chemical or enzymatic degradation processes before it reaches the target tissue.

Therefore, further object of the present invention is irisin included in liposomes for its use as a medicament. In other words, the present invention relates, in one aspect thereof, to liposomes comprising irisin for their use as a medicament.

For example, irisin included in liposomes (i.e., liposomes comprising irisin) may be used in the treatment and/or prevention of osteoporosis, sarcopenia, energy metabolism disorders, diseases of the cardiovascular system, neurodegenerative diseases, diabetes, obesity, kidney diseases and metabolic diseases.

Advantageously, liposomes have no limitations of use in terms of possible routes of administration. In fact, liposomes represent a biologically safe system since they are made of phospholipids, which are natural components of all cell membranes.

In embodiments, irisin included, preferably encapsulated, in liposomes may be administered sublingually and/or subcutaneously and/or intradermally, preferably by the use of a dermal gel, and/or nasally, preferably by the use of a nasal spray. In other words, liposomes comprising irisin can be administered sublingually and/or subcutaneously and/or intradermally, preferably by the use of a dermal gel, and/or nasally, preferably by the use of a nasal spray.

The present invention demonstrates that irisin can be expressed in organisms of plant origin and that this protein is biologically active, with an effect which may be superimposed on that induced by commercial irisin used at the same concentration. Therefore, the expression of irisin in plants such as Nicotiana benthamiana has a number of advantages over traditional systems which use bacteria (prokaryotic cells) or mammalian cells (eukaryotic cells) as systems for the production of recombinant proteins. For example, plants can be cultivated easily on a large scale, are free of immunogenic bacterial endotoxins and pathogens. Furthermore, the use of plant material has significantly lower costs than known methods, thus allowing the production of a large amount of irisin at low cost, enabling its use for therapeutic purposes. Furthermore, the present invention enables the rapid advancement of the studies in the medical field, which require high amounts of the protein.

The invention may be even better understood thanks to the illustrative, non-limiting examples described in the following Experimental Section and accompanied by the FIGS. 1-6.

### BRIEF DESCRIPTION OF THE FIGURES

FIG. 1 shows the different steps of the preparation of the expression vector comprising a nucleotide sequence coding for irisin. The pBIΩ-IRS vector, once obtained, is inserted into bacteria of the Agrobacterium genus.

FIG. 2 shows the results of the quantization of the irisin expression by Western blot.

FIG. 3 shows the results of the quantization of the irisin expression by ELISA assay.

FIG. 4 shows the results obtained in an in vitro assay of phosphorylation of murine osteoblast MAP kinase to verify the biological effect of irisin contained in N. benthamiana extracts.

FIG. 5 shows the results obtained in an assay performed to evaluate the biological activity of irisin expressed and purified from plant cells, which is included in liposomes.

FIG. 6 shows the map of the modified pJL-TRBO expression vector.

### EXPERIMENTAL SECTION

### Example 1—Preparation of the Expression Vector Comprising a Nucleotide Sequence Coding for Irisin and its Insertion into Agrobacterium

The experimental work was carried out by using mainly the Nicotiana benthamiana plant; however, the process is reproducible in other types of plant organisms such as Cannabis sativa, Arthrospira platensis, Chlorella, Arabidopsis, maize, rice, soybean, canola, alfalfa, sunflower, sorghum, wheat, cotton, peanut, tomato, potato, lettuce and chili pepper.

The nucleotide sequences coding for the portion of the protein corresponding to irisin, which are optimized for the plant codon, were engineered to have the construct expressed in the plant tissues. Based on the amino acid sequence of irisin (112 amino acids, SEQ. ID. NO. 1), a nucleotide sequence was synthesized and optimized for expression in Nicotiana benthamiana (SEQ. ID. NO. 2). The nucleotide sequence SEQ. ID. NO. 2 includes a restriction site for the BamHI enzyme (the first underlined 6 nucleotides) and a restriction site for the SmaI enzyme (the last underlined 6 nucleotides). The nucleotide sequence SEQ. ID. NO. 2 further includes two stop codons (the 6 nucleotides immediately preceding the restriction site for the SmaI enzyme).

All of the synthetic genes were optimized for expression codons in N. benthamiana by using the Optimum-Gene™ algorithm (GenScript, Piscataway, NJ).

### Amino Acid Sequence of the Native Irisin:

(SEQ. ID. NO. 1)
DSPSAPVNVTVRHLKANSAVVSWDVLEDEVVIGFAISQQKKDVRMLRFIQE
VNTTTRSCALWDLEEDTEYIVHVQAISIQGQSPASEPVLFKTPREAEKMAS
KNKDEVTMKE

Polynucleotide Sequence for the Expression of Irisin in Nicotiana benthamiana:

(SEQ. ID. NO. 2)
GGATCCGATTCTCCTTCAGCTCCAGTTAATGTTACAGTTAGACATCTTAAG
GCTAATTCTGCTGTTGTTTCATGGGATGTTTTGGAAGATGAGGTTGTTATT
GGTTTTGCTATCTCTCAACAGAAGAAAGATGTTAGAATGCTTAGGTTCATC
CAAGAAGTTAACACTACAACTAGGTCTTGTGCTCTTTGGGATTTGGAAGAG
GATACAGAGTACATCGTTCATGTTCAGGCTATCTCAATCCAAGGACAGTCT
CCTGCTTCAGAACCAGTTTTGTTTAAAACTCCTAGGGAGGCTGAGAAAATG
GCAAGTAAAAACAAGGATGAGGTGACAATGAAAGAGTGATAGCCCGGG

### Amino Acid Sequence Codified by the Polynucleotide Sequence Set Forth Above:

(SEQ. ID. NO. 3)
DSPSAPVNVTVRHLKANSAVVSWDVLEDEVVIGFAISQQKKDVRMLRFIQE
VNTTTRSCALWDLEEDTEYIVHVQAISIQGQSPASEPVLFKTPREAEKMAS
KNKDEVTMKE

As can be observed, the amino acid sequence of the recombinant irisin produced according to the present invention (SEQ. ID. NO. 3) is the same as the amino acid sequence of native irisin (SEQ. ID. NO. 1).

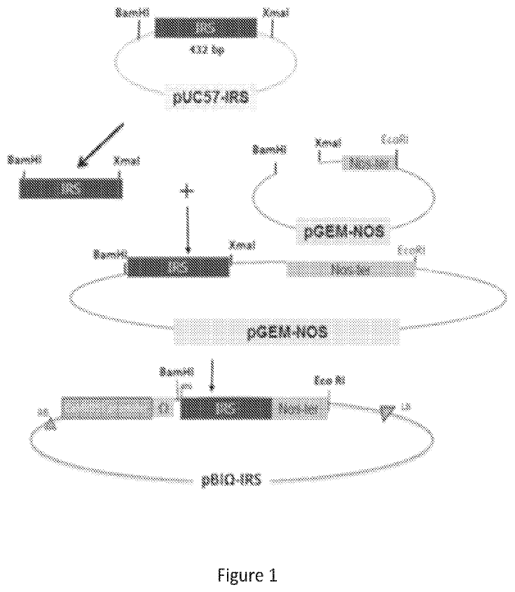

The cloning of the synthetic gene coding for irisin having sequence SEQ. ID. NO. 2 in the pBIΩ expression vector is schematized in FIG. 1. The construct containing the nucleotide sequence coding for irisin was cloned by GeneScript and inserted into the pUC57 plasmid by using two restriction sites (the BamHI site at 5′ and the XmaI site at 3′). From the plasmid pUC57-IRS, containing the synthetic gene coding for irisin (i.e., the sequence SEQ. ID. NO. 2), the insert (IRS, corresponding to the sequence coding for irisin) was purified by cutting with the BamHI and XmaI restriction enzymes. Then the gene was cloned into an intermediate vector. A binary vector, capable of replication in both E. coli and Agrobacterium tumefaciens, was used for such step. The step in E. coli was performed to increase transcription and amplification efficiency. The need to use a step in the intermediate vector, before moving to the vector to be transferred into the agrobacterium, is also driven by the lack of useful restriction sites in the final vector. Therefore, pGEM-NOS, a commercial E. coli vector, was used as the intermediate vector within which a plant terminator, Nos-ter, was cloned, which is necessary for the integration of the final vector into the plant, that is, for the integration of the recombinant DNA within the plant genome.

In the next steps, the final construct was purified from E. coli (by a currently commercially available kit) and was electroporated into agrobacterium LBA4404 (i.e., Agrobacterium tumefaciens strain LBA4404) by using the pBIΩ-IRS expression vector containing, at position 5′, a transcriptional enhancer (CaMV35S promoter, i.e., a viral promoter) to increase even more the transcript levels.

In the same vector (pBIΩ), the P19 gene was cloned, which is essential for inhibiting the silencing of the irisin gene. The vector containing P19 was inserted into Agrobacterium but separately from the vector containing the gene for irisin. Such process is necessary to inhibit the fragmentation and silencing of the foreign gene, i.e., the irisin gene, by plant cells which, in the presence of an overproduced foreign transcript, i.e., produced in significantly greater amounts than the transcripts derived from endogenous genes of the host plant, in this case the transcript deriving from the gene for irisin, could activate such defense mechanisms. Therefore, cultures of agrobacterium LBA4404 transformed with binary vector containing the gene for irisin and the enhancer, and cultures of agrobacterium transformed with binary vector containing the gene P19 and the enhancer, were cultivated separately and then combined before agro-infiltration. Finally, cultures of Agrobacterium tumefaciens LBA4404 containing the two constructs were used to agro-infiltrate the plants.

### Example 2—Agro-Infiltration with Agrobacterium Tumefaciens LBA4404 of Nicotiana benthamiana Leaves

The transient expression in the plant leaves was obtained by vacuum infiltration. The expression is defined “transient”, as it affects only infected leaves. The Agrobacterium tumefaciens (LBA4404) clones harboring the constructs described above were cultured separately, the bacteria were sedimented by centrifugation at 4000 g and resuspended in infiltration buffer (10 mM MES, 10 mM MgSO₄, pH 5.8). The Agrobacterium suspensions harboring the different vectors were used separately or mixed together to reach the final optical density (OD600) of 0.5 for each construct. Six-week-old hydroponically cultivated N. benthamiana plants (at the 6-7 leaf stage) were infiltrated by fully submerging each plant in the solution containing Agrobacterium inside a dryer. Vacuum was applied reaching about 10 mm Hg and then released rapidly. The infiltration was confirmed visually by observing the infiltrated areas as translucent. The plants were then placed in the greenhouse and the leaf sampling was performed at different and predetermined times, from 2 to 7 days after the infiltration (2-7 DPI). Leaves of the same age (from the “middle leaf” position, typically leaf 4 and 5 from the bottom) from three individual plants were collected and stored in liquid N₂. In the same experiments, plants agro-infiltrated with the same vectors lacking the gene coding for irisin were cultivated with the same experimental procedures and used as a negative control. To verify the expression and subsequent purification of irisin, batches of 40 g of agro-infiltrated leaves of all ages were collected, frozen immediately in liquid N₂ and stored at −80° C. The samples were then subjected to extraction procedure in PBS, supplemented with protease inhibitors and used to test and quantify the irisin expression by Western blot and ELIA analyses.

### Example 3—Quantification of Irisin Expression by Western Blot and ELISA

The leaf tissue (100 mg) was ground in liquid N₂ and homogenized in 500 μl of pH 7.2 phosphate-buffered saline (PBS) containing a protease inhibitor cocktail (Complete™; Roche, Mannheim, Germany). After centrifugation at 20000 g at 4° C., for 30 min, the supernatant was recovered and quantified for the total content of soluble proteins by using the DC™ Protein Assay (Bio-Rad, California, USA). Leaves from the same position on the plants (leaf number 4 and 5 from the bottom) were collected after 2, 3, 4, 5, 6 and 7 days after the infiltration (DPI) and used to test the expression by Western blot analysis by using an anti-FNDC5 antibody (Abcam-ab 131390). In the same experiments, agro-infiltrated plants with the same vectors lacking the gene coding for irisin were cultivated with the same experimental procedures and used as a negative control.

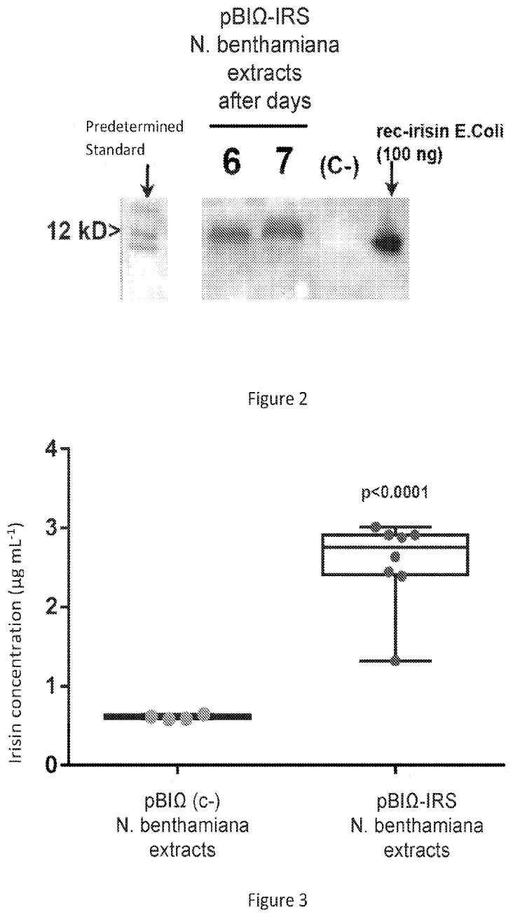

The Western blot analysis under reducing conditions of the leaf extracts showed the presence of bands of the molecular weight of 12 kd corresponding to irisin, as demonstrated by the band generated by the recombinant protein produced in E. Coli, currently commercially available and used as a positive control (Adipogen-AG-40B-0103-C010) (FIG. 2). The same band was not matched in the negative control.

The maximum expression of irisin, denoted by the intensity of the 12-kDa band, was detected at 7 days after infiltration (FIG. 2).

An ELISA test (AG-45A-0046YEK-KI01, Adipogen) was performed to quantify irisin present in the leaf extracts. The Phoenix Pharmaceutical ELISA kit is designed to measure the irisin concentration based on the principle of competitive enzyme immunoassay. The 96-well plate of this kit is pre-coated with recombinant irisin. The polyclonal antibody specific for irisin reacts competitively, in the irisin-coated plate, with the recombinant irisin added at known concentrations in the standard curve and with samples of the leaf extracts with unknown concentration of irisin. The resulting color intensity will be inversely proportional to the amount of irisin in the standard irisin solution or leaf extracts. This is due to competition for the binding with the primary antibody between the irisin in the standard curve or leaf extracts and the irisin molecule present in the coating of the 96-well plate. The standard curve is made by interpolating the optical density points measured as a function of various known concentrations of standards. The irisin concentrations (average 2.563 μg/ml±0.55 SD) in the leaf extracts were determined by extrapolation based on the standard curve (FIG. 3).

Furthermore, the average levels were 0.5 mg/100 g of fresh leaf tissue, which corresponded to about 0.6% of the total content of soluble protein, respectively.

### Example 4—Biological Effect of Irisin Contained in the N. Benthamiana Extracts on Murine Osteoblasts In Vitro

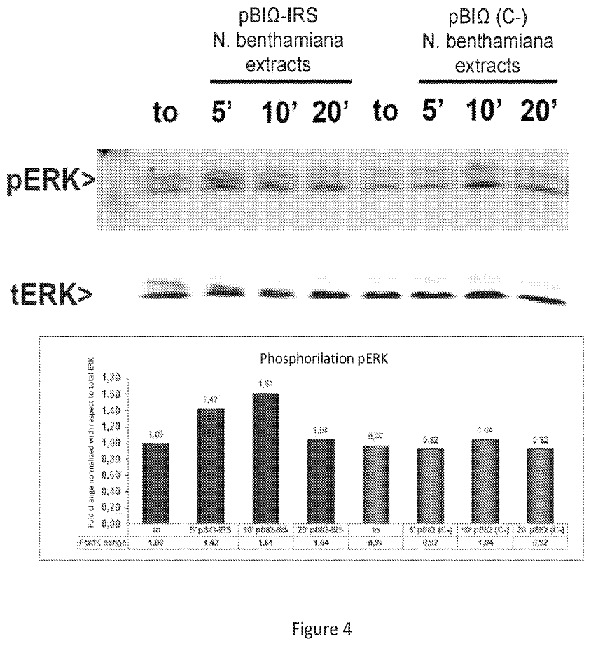

To demonstrate that irisin expressed and purified from the plant cells is biologically active, the action of the extract was evaluated in vitro. In particular, referring to the fact that irisin is capable of activating the MAP kinase ERK in the osteo-progenitor cells, the osteoblasts, we performed a test by stimulating the osteoblasts for 5, 10 and 20 min with extracts of N. benthamiana leaves agro-infiltrated with pBIΩ-IRS containing 100 ng/ml of irisin or with the corresponding negative control (C-) pBIΩ not containing the synthetic gene coding for irisin having sequence SEQ. ID. NO. 2 (FIG. 4). At the end of the stimuli, the osteoblasts were lysed with the lysis buffer [50 mM Tris (Tris(hydroxymethyl)aminomethane)-HCl (pH 8.0), 150 mM HCl, 5 mM ethylenediaminetetraacetic acid, 1% NP40 and 1 mM phenylmethyl sulfonyl fluoride]. The protein concentration was measured with the DC™ Protein Assay (Bio-Rad, California, USA). 20 μg of cellular proteins were subjected to SDS-polyacrylamide gel electrophoresis (SDS-PAGE) and then transferred onto nitrocellulose membranes (Millipore, Massachusetts, USA). The blots were incubated over-night at 4° C. by using anti-pospho ERK (pERK) primary antibody (Santa Cruz Biotechnology) and anti-total Erk (tERK) (Santa Cruz Biotechnology). Then the membranes were incubated for two hours at room temperature with the secondary antibodies labeled with IRDye (680/800 CW) (LI-COR Biosciences). For the immuno-detection, the Odyssey infrared imaging system (LI-COR Corp., Lincoln, NE) was used. All data were normalized with respect to tERK and calculated as fold change (i.e., the number of times the signal is amplified) with respect to time zero (t0).

Our results show that 5 min stimulation with N. benthamiana leaf extracts agro-infiltrated with pBIΩ-IRS activates the phosphorylation of MAP kinase ERK in the osteoblasts. The phosphorylation of ERK remains active up to 10 min, then shuts down 20 min after the stimulus. In parallel, the N. benthamiana leaves extracts agro-infiltrated with the negative control (C-) pBIΩ not containing the synthetic gene coding for irisin of sequence SEQ. ID. NO. 2 are unable to stimulate the phosphorylation of MAP kinase ERK (FIG. 4). The data obtained from this biological activity test show that irisin produced by N. benthamiana leaves is effective in directly activating the intracellular signal triggered in the osteo-forming cells, exactly as already observed for irisin produced not in plant material but in E. Coli.

### Example 5—Preparation of Liposomal Irisin Incorporated into Lecithin/Chitosan Nanoparticles (NLC) and Biological Testing on Murine Osteoblastic Lines

For the preparation of liposomal Irisin, a solution of soy lecithin in methanol was used to which different concentrations of powdered Irisin were added. The mixture thus obtained was subjected to sonication in order to promote the encapsulation of irisin within the liposome.

A chitosan solution, which promotes the transmucosal permeation of substances, was prepared in parallel. The organic lecithin/irisin solution was added to such solution.

This way, the liposomal NLC nanoparticles embedded with irisin (NLC-I) are produced.

After verifying the efficiency of the encapsulation, the NLC-Is, upon sterilization by filtration, were used for biological tests at the final concentration of 100 ng/ml irisin.

In particular, cultures of murine osteoblastic cell lines were stimulated, for 8 hours, with liposomal irisin and non-encapsulated irisin, as a control, at the same concentration.

Next, cell extracts were prepared for the analysis of the gene levels of two transcription factors, the one regulating osteoblast formation, ATF4, and the one regulating mitochondrial biogenesis, TFAM, already known to be modulated by non-encapsulated (non-liposomal) irisin.

The results showed that the osteoblasts treated with liposomal irisin undergo an increase in the mRNA levels for both transcription factors, ATF4 and TFAM, in a way comparable to that obtained with non-liposomal irisin stimulation.

### Example 6—Biological Effect of Irisin Contained in the N. Benthamiana Extracts and Encapsulated in Liposomes on Murine Muscle Cells In Vitro

To demonstrate that irisin expressed and purified from the plant cells is biologically active also when included in liposomes, the gene expression of the mitochondrial transcription factor A (Tfam), an irisin-modulated transcription factor that plays a key role in the process of mitochondriogenesis, was evaluated in murine muscle cells. More specifically, the muscle cells were treated for 8 hours with liposomes containing N. benthamiana leave extracts agro-infiltrated with pBIΩ-IRS or with liposomes containing the corresponding negative control (C-) pBIΩ not containing the gene coding for irisin (sequence SEQ. ID. NO. 2).

RNA was extracted from muscle cells with RNeasy Mini Kit (Qiagen, Hilden, Germany) by using centrifugation columns according to the manufacturer's instructions. Reverse transcription was performed with iScript Reverse Transcription Supermix (Bio-Rad, Hercules, CA, USA) in the MyCycler thermal cycler (Bio-Rad, Hercules, CA, USA) according to the manufacturer's instructions. Quantitative real-time Polymerase Chain Reaction (qPCR) was performed by using SsoFast EvaGreen Supermix (Bio-Rad, Hercules, CA, USA) with CFX96 real-time thermal cycler (Bio-Rad, Hercules, CA, USA) for 40 cycles (denaturation, 95° C. for 5 s; annealing/extension, 60° C. for 10 s) after an initial 30-second step for the enzyme activation at 95° C. Primer-BLAST was used to identify the primers of interest. Gapdh was selected as the housekeeping gene because it was stably expressed in all samples. The sequences of the primers used are as follows:

Gapdh
Forward primer:
(SEQ. ID NO. 4)
acaccagtagactccacgaca
Reverse primer:
(SEQ. ID NO. 5)
acggcaaattcaacggcacag
Tfam
Forward primer:
(SEQ. ID NO. 6)
taggcaccgtattgcgtgag
Reverse primer:
(SEQ. ID NO. 7)
cagacaagactgatagacgaggg.

All data were normalized with respect to Gapdh and calculated as fold change (i.e., the number of times the signal is amplified) with respect to the negative control (C-) pBIΩ.

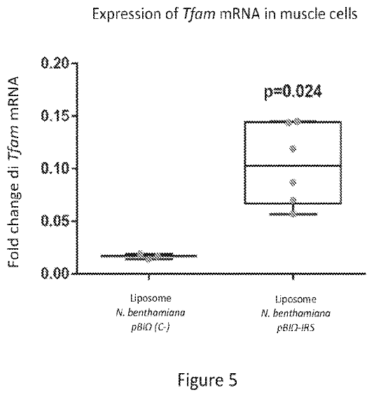

As shown in FIG. 5, the results obtained demonstrate that 8 hours of stimulation with liposomes containing N. benthamiana leaf extracts agro-infiltrated with pBIΩ-IRS are effective in increasing the expression of Tfam mRNA in muscle cells, compared with the liposomes containing N. benthamiana leaf extracts agro-infiltrated with the negative control (C-) pBIΩ2 not containing the gene coding for irisin. The data obtained from this biological activity test show that irisin produced by N. benthamiana leaves and encapsulated in the liposomes is effective in the up-regulation of the mitochondriogenesis in muscle cells, exactly as already observed for irisin produced not in plant material but in E. Coli.

### Example 7—Modified pJL-TRBO Expression Vector and Synthetic Gene Including Irisin Sequence and Additional Elements

A synthetic gene was designed that includes the sequence coding for irisin, fused with additional elements that enable the expression of the protein in the plant. The resulting cDNA coded for a polypeptide constituted by: I) an N-terminal signal peptide for the secretion from the disulfide isomerase of Medicago sativa alfalfa, II) the protein irisin, III) the thrombin cleavage site followed by IV) a 8-His tag and V) a KDEL-terminal tag that allows the retention of the protein in the endoplasmic reticulum. The overall sequence of the synthetic gene and the protein it codes are shown below (sequences SEQ. ID NO. 8 and SEQ. ID NO. 9, respectively).

(SEQ. ID NO. 8)
atggctaagaatgttgctatttttggacttcttttttctcttcttgttctt
gttccatctcaaatttttgctgattctccttcagctccagttaatgttaca
gttagacatcttaaggctaattctgctgttgtttcatgggatgttttggaa
gatgaggttgttattggttttgctatctctcaacagaagaaagatgttaga
atgcttaggttcatccaagaagttaacactacaactaggtcttgtgctctt
tgggatttggaagaggatacagagtacatcgttcatgttcaggctatctca
atccaaggacagtctcctgcttcagaaccagttttgtttaaaactcctagg
gaggctgagaaaatggcaagtaaaaacaaggatgaggtgacaatgaaagag
cttgttccaagaggatctcatcatcatcatcatcatcatcat
aaggatgaactt

The polynucleotide sequence SEQ. ID. NO. 8 includes the polynucleotide sequence of irisin. Highlighted in bold is the sequence coding for the N-terminal signal peptide for the secretion from the disulfide isomerase of Medicago sativa alfalfa; highlighted with single underline is the sequence coding for the thrombin cleavage site; highlighted with double underline is the sequence coding for the 8-His tag; and highlighted with the combination of bold and single underline is the sequence coding for the KDEL tag.

(SEQ. ID NO. 9)
MAKNVAIFGLLFSLLVLVPSQIFADSPSAPVNVTVRHLKANSAVVSWDVL
EDEVVIGFAISQQKKDVRMLRFIQEVNTTTRSCALWDLEEDTEYIVHVQA
ISIQGQSPASEPVLFKTPREAEKMASKNKDEVTMKELVPRGSHHHHHHHH
KDEL

The amino acid sequence SEQ. ID. NO. 9 includes the amino acid sequence of irisin. Highlighted in bold is the N-terminal signal peptide for the secretion from the disulfide isomerase of Medicago sativa alfalfa; highlighted with single underline is the thrombin cleavage site; highlighted with double underline is the 8-His tag; and highlighted with the combination of bold and single underline is the KDEL tag.

The synthetic genes were optimized for the expression in Nicotiana benthamiana and cloned into a modified version of the pJL-TRBO plasmid vector (i.e., into a modified pJL-TRBO plasmid).

The modified pJL-TRBO plasmid is the pJL-TRBO plasmid in which the restriction sites pBluescriptKS, Pvu I, GFP-R, BstZ17 I and GFP-F have been removed, and into which the restriction sites Sal I, Mlu I, Nhe I, Bmt I, Eco53k I, Sac I, Nco I, Nru I, Asc I-BssH II, ApaL I, AsiS I, Swa I, Kas I, Nar I, Sfo I, PluT I, Avr II have been inserted.

The modified pJL-TRBO plasmid was obtained from the original pJL-TRBO plasmid, currently commercially available and sold by Addgene (Watertown, MA, USA), by techniques known, per se, in the art.

The modified pJL-TRBO plasmid has a length of 10659 base pairs.

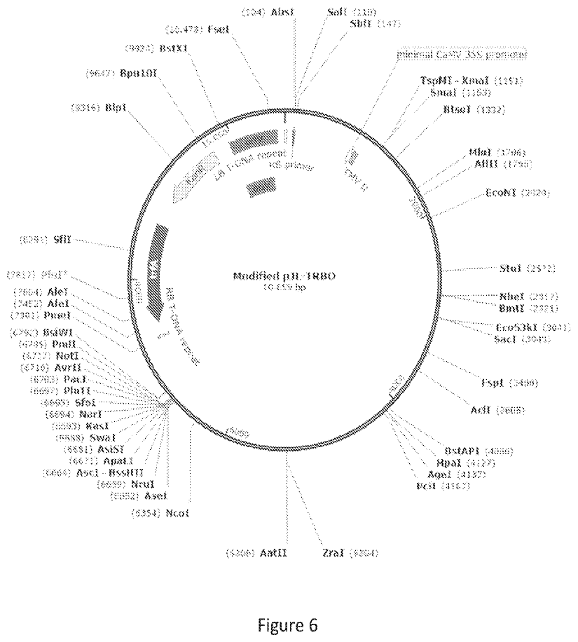

The modified pJL-TRBO plasmid is shown in FIG. 6.

As it may be observed from FIG. 6, the modified pJL-TRBO plasmid includes the 35S promoter from CAMV (cauliflower mosaic virus) that allows the expression of high levels of proteins in the plant, the replication origin site, the gene for kanamycin antibiotic resistance, the gene of the replication initiation protein (trfA), the 5′-leader sequence (named Omega) of the tobacco mosaic virus (TMV), a “Left border repeat of T-DNA” region, a “Right border repeat of T-DNA” region and the KS primer sequence used for the amplification and sequencing of the right end of the gene. Once recognized by the plasmid in Agrobacterium, the region between the T-DNA boundary repeats is transferred to the plant cells.

In particular in FIG. 6, the term “minimal CaMV 35S promoter” “is meant to refer to the 35S promoter of CAMV (cauliflower mosaic virus) that allows the expression of high levels of protein in the plant; the term “OriV” is meant to refer to the site of origin of the replication; the term “KanR” is meant to refer to “Kanamycin resistance”; the term “trfA” is meant to refer to “replication initiation protein”; the term “TMV Q” is meant to refer to the 5′-leader sequence (called Omega) of the tobacco mosaic virus (TMV); the term “LB T-DNA repeat” is meant to refer to “Left border repeat of T-DNA”; the term “RB T-DNA repeat” is meant to refer to “Right border repeat of T-DNA”; and the term “KS primer” is meant to refer to the primer used for the amplification and sequencing of the right end of the gene.

The final genetic construct was used to transform electro-competent cells of Agrobacterium tumefaciens LBA4404 and generate a glycerol stock bank of the transformed bacteria.

Subsequently, large cultures of engineered A. tumefaciens cells were used for the agro-infiltration of N. benthamiana plants, allowing transient expression of the protein of interest (i.e., irisin) in plant leaves.

The presence of the His tag allowed the purification of the protein by IMAC (immobilized metal ion affinity chromatography) and the subsequent thrombin treatment ensured the removal of the tag from the purified irisin.

At the end of the test, irisin expression levels were at or above 5 mg of protein per gram of fresh leaf tissue.

1. A process for the production of irisin, comprising the following steps:
a) providing at least one expression vector comprising one nucleotide sequence coding for said irisin;
b) inserting said expression vector into at least one first bacterium of the Agrobacterium genus, thus obtaining a first bacterium of the Agrobacterium genus comprising said expression vector;
c) treating at least one plant material with said first bacterium comprising said expression vector, thus obtaining one treated plant material;
d) cultivating said treated plant material, so that said treated plant material expresses said irisin;
e) extracting said irisin from said treated plant material.

2. The process according to claim 1, further comprising, before said step a), a step wherein an amplifying vector comprising said nucleotide sequence coding for irisin is inserted into Escherichia coli and amplified through cultivation of said Escherichia coli.

3. The process according to claim 1, wherein said irisin is non-glycosylated irisin.

4. The process according to claim 1, wherein said irisin is partially glycosylated and wherein said process further comprises a step of removing the glycosylation, thus obtaining non-glycosylated irisin.

5. The process according to claim 1, wherein said plant material is selected from a plant, or at least one part of said plant, or at least one cell of said plant.

6. The process according to claim 5, wherein said plant material is a plant selected from tobacco, preferably Nicotiana benthamiana, Cannabis sativa, Arthrospira platensis, Chlorella, Arabidopsis, corn, rice, soy, canola, alfalfa, sunflower, sorghum, wheat, cotton, peanut, tomato, potato, lettuce and chili pepper, or at least one part of said plant, or at least one cell of said plant.

7. The process according to claim 1, wherein said step c) further comprises the step of treating said at least one plant material with at least one second bacterium of the Agrobacterium genus, said second bacterium comprising an expression vector comprising a gene adapted to prevent the silencing of the expression of said irisin in said treated plant material.

8. The process according to claim 7 wherein, in said step c), said at least one plant material is treated with a mixture comprising said first bacterium comprising said expression vector comprising a nucleotide sequence coding for said irisin, and said second bacterium comprising a gene adapted to prevent the silencing of the expression of said irisin in said treated plant material.

9. The process according to claim 1, wherein said bacterium of the Agrobacterium genus is Agrobacterium tumefaciens.

10. The process according to claim 7, wherein said gene adapted to prevent the silencing of the expression of said irisin is the P19 gene.

11. The process according to claim 1, wherein said expression vector is the pJL-TRBO plasmid in which the restriction sites pBluescriptKS, Pvu I, GFP-R, BstZ17 I and GFP-F have been removed, and into which the restriction sites Sal I, Mlu I, Nhe I, Bmt I, Eco53k I, Sac I, Nco I, Nru I, Asc I-BssH II, ApaL I, AsiS I, Swa I, Kas I, Nar I, Sfo I, PluT I, Avr II have been inserted.

12. The process according to claim 1, wherein said nucleotide sequence coding for said irisin is included in a synthetic gene comprising, in addition to said sequence coding for said irisin, a polynucleotide sequence coding for an N-terminal signal peptide for the secretion from the disulfide isomerase of Medicago sativa alfalfa; a sequence coding for the thrombin cleavage site, followed by a sequence coding for a tail of 8 histidine residues and a sequence coding for a KDEL-terminal tail, wherein the nucleotide sequence coding for irisin is between said polynucleotide sequence coding for an N-terminal signal peptide for the secretion from the disulfide isomerase of Medicago sativa alfalfa and said sequence coding for the thrombin cleavage site.

13. The process according to claim 1, wherein said nucleotide sequence coding for said irisin is
(SEQ. ID. NO. 2)
GGATCCGATTCTCCTTCAGCTCCAGTTAATGTTACAGTTAGACATCTTAA
GGCTAATTCTGCTGTTGTTTCATGGGATGTTTTGGAAGATGAGGTTGTTAT
TGGTTTTGCTATCTCTCAACAGAAGAAAGATGTTAGAATGCTTAGGTTCA
TCCAAGAAGTTAACACTACAACTAGGTCTTGTGCTCTTTGGGATTTGGAA
GAGGATACAGAGTACATCGTTCATGTTCAGGCTATCTCAATCCAAGGACA
GTCTCCTGCTTCAGAACCAGTTTTGTTTAAAACTCCTAGGGAGGCTGAGA
AAATGGCAAGTAAAAACAAGGATGAGGTGACAATGAAAGAGTGATAGC
CCGGG.

14. An irisin included in liposomes.

15. The irisin included in liposomes according to claim 14, wherein said irisin is non-glycosylated irisin.

16. The irisin included in liposomes according to claim 14, wherein said irisin is encapsulated within said liposomes.

17. The irisin included in liposomes, wherein said liposomes comprise lecithin and chitosan.

18. The irisin included in liposomes for its use as a medicament.

19. The irisin included in liposomes according to claim 18, for its use in the treatment and/or prevention of one or more diseases selected from osteoporosis, sarcopenia, disorders of the energy metabolism, diseases of the cardiovascular system, neurodegenerative diseases, diabetes, obesity, renal diseases and metabolic diseases.

20. The irisin for its use according to claim 18 or 19, wherein said irisin included in liposomes is administered by sublingual and/or subcutaneous and/or intradermal route through a dermal gel, and/or by nasal route through a nasal spray.

21. A synthetic gene for the expression of irisin in plants, comprising: a polynucleotide sequence coding for an N-terminal signal peptide for the secretion from the disulfide isomerase of Medicago sativa alfalfa; a sequence coding for irisin, a sequence coding for the thrombin cleavage site, a sequence coding for a tail of 8 histidine residues and a sequence coding for a KDEL-terminal tail.


SEQUENCE LISTING (9 sequences)

SEQ ID NO: 1 - protein, 112 aa, Homo sapiens
DSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKE

SEQ ID NO: 2 - DNA, 354 bp, synthetic construct
  misc_feature 1..354: note = sequence encoding for irisin
  source 1..354: mol_type = other DNA
ggatccgattctccttcagctccagttaatgttacagttagacatcttaaggctaattctgctgttgtttcatgggatgttttggaagatgaggttgttattggttttgctatctctcaacagaagaaagatgttagaatgcttaggttcatccaagaagttaacactacaactaggtcttgtgctctttgggatttggaagaggatacagagtacatcgttcatgttcaggctatctcaatccaaggacagtctcctgcttcagaaccagttttgtttaaaactcctagggaggctgagaaaatggcaagtaaaaacaaggatgaggtgacaatgaaagagtgatagcccggg

SEQ ID NO: 3 - protein, 112 aa, synthetic construct; identical to SEQ ID NO: 1
  REGION 1..112: note = Recombinant Irisin

SEQ ID NO: 4 - DNA, 21 bp, synthetic construct
  source 1..21: mol_type = other DNA
  misc_feature 1..21: note = Gapdh - Primer forward
acaccagtagactccacgaca

SEQ ID NO: 5 - DNA, 21 bp, synthetic construct
  source 1..21: mol_type = other DNA
  misc_feature 1..21: note = Gapdh - Prime reverse
acggcaaattcaacggcacag

SEQ ID NO: 6 - DNA, 20 bp, synthetic construct
  source 1..20: mol_type = other DNA
  misc_feature 1..20: note = Tfam - Primer forward
taggcaccgtattgcgtgag

SEQ ID NO: 7 - DNA, 23 bp, synthetic construct
  source 1..23: mol_type = other DNA
  misc_feature 1..23: note = Tfam - Primer reverse
cagacaagactgatagacgaggg

SEQ ID NO: 8 - DNA, 462 bp, synthetic construct
  source 1..462: mol_type = other DNA
  misc_feature 1..462: note = synthetic gene
atggctaagaatgttgctatttttggacttcttttttctcttcttgttcttgttccatctcaaatttttgctgattctccttcagctccagttaatgttacagttagacatcttaaggctaattctgctgttgtttcatgggatgttttggaagatgaggttgttattggttttgctatctctcaacagaagaaagatgttagaatgcttaggttcatccaagaagttaacactacaactaggtcttgtgctctttgggatttggaagaggatacagagtacatcgttcatgttcaggctatctcaatccaaggacagtctcctgcttcagaaccagttttgtttaaaactcctagggaggctgagaaaatggcaagtaaaaacaaggatgaggtgacaatgaaagagcttgttccaagaggatctcatcatcatcatcatcatcatcataaggatgaactt

SEQ ID NO: 9 - protein, 154 aa, synthetic construct
  REGION 1..154: note = synthetic polypeptide
MAKNVAIFGLLFSLLVLVPSQIFADSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKELVPRGSHHHHHHHHKDEL